(19) United States
(12) Patent Application Publication
(10) Pub. No.: US 2026/0365985 A1
(43) Pub. Date: Nov. 19, 2026

(54) COMPOSITION CONTAINING CELL OR CELL PRODUCT, AND METHOD FOR PRODUCING CELL OR CELL CLUSTER, AND/OR CELL PRODUCT

(71) Applicant: CellFiber Co., Ltd., Koto-ku, Tokyo (JP)

(72) Inventors: Kazuhiro IKEDA, Koto-ku, Tokyo (JP); Tomoyuki KANEIWA, Koto-ku, Tokyo (JP); Koshi OYANAGI, Koto-ku, Tokyo (JP); Risa SAKATE, Koto-ku, Tokyo (JP)

(73) Assignee: CellFiber Co., Ltd., Koto-ku, Tokyo (JP)

(21) Appl. No.: 19/133,557

(22) PCT Filed: Nov. 14, 2023
(86) PCT No.: PCT/JP2023/040955

§ 371 Date: May 28, 2025

(30) Foreign Application Priority Data
Nov. 29, 2022 (JP) 2022-190850

(51) Int. Cl. C12N 5/077; C12N 5/071; C12N 5/09

(52) U.S. Cl. CPC C12N 5/0656; C12N 5/0686; C12N 5/0693; C12N 2509/10; C12N 2533/56; C12N 2533/74

(57) ABSTRACT

Provided is a method for producing cells or cell cluster and/or cell products, the method facilitating removal of hydrogel-derived substances. The method for producing a cell or a cell cluster and/or a cell product, comprises: culturing a cell or a cell cluster in a core material to obtain a cell, a cell cluster and/or a cell product by using a capsule including the core material and a shell covering the core material; and physically breaking or cutting at least the shell to collect the cell or the cell cluster and/or the cell product.

### TECHNICAL FIELD

The present invention relates to a composition containing a cell or a cell product, and a method for producing a cell or a cell cluster, and/or a cell product.

### BACKGROUND ART

Currently, attempts have been made to culture various cells in various fields such as developmental biology, drug discovery, and regenerative medicine. These cells are typically cultured two-dimensionally on a surface of a culture plate. Such culture is performed, for example, not only for the purpose of cell proliferation, but also for the purpose of antibody production, viral vector production, or biologically active substance production. However, in order to culture a large amount of cells by such a two-dimensional culture method using a culture plate, a large number of culture plates are required.

On the other hand, a technique for culturing cells in a three-dimensional or pseudo-three-dimensional culture environment using a scaffold such as a porous membrane or a hydrogel is known. Patent Literature 1 to 6 below disclose the culturing of cells inside a hydrogel such as a tubular alginate gel. In addition, Patent Literature 7 discloses that cells are cultured inside a hydrogel such as a substantially spherical alginate gel. The cells contained in a hydrogel in this manner can be cultured in a reactor containing a liquid medium in a state of being enclosed in the hydrogel.

### CITATION LIST

### Patent Literature

Patent Literature 1: WO 2011/046105 A1
Patent Literature 2: JP 2017-099303 A
Patent Literature 3: WO 2017/091662 A1
Patent Literature 4: WO 2018/098295 A1
Patent Literature 5: WO 2019/178549 A1
Patent Literature 6: WO 2020/032221 A1
Patent Literature 7: WO 2018/096277 A1

### SUMMARY

In a case where a hydrogel such as an alginate gel is used, the alginate gel is dissolved using a chelating agent such as EDTA when cells are collected from the hydrogel.

However, when dissolving the hydrogel, a hydrogel dissolution product (for example, alginic acid) will be present in the solution. In order to remove the hydrogel dissolution product from a composition containing a cell or a cell product collected from the inside of the hydrogel, a complicated treatment may be required. Therefore, a method for producing a cell or a cell cluster and/or a cell product from which a hydrogel-derived substance (for example, a hydrogel dissolution product) can be easily removed, and a composition containing a cell or a cell product from which the hydrogel-derived substance has been removed at a relatively high level are desired.

In one aspect, a method for producing a cell or a cell cluster and/or a cell product, comprises: culturing a cell or a cell cluster in a core material to obtain a cell, a cell cluster and/or a cell product by using a capsule including the core material and a shell covering the core material; and physically breaking or cutting at least the shell to collect the cell or the cell cluster and/or the cell product.

In one aspect, a composition containing a cell or a cell product is produced by culturing a cell or a cell cluster in a core material to obtain a cell, a cell cluster and/or a cell product by using a capsule including the core material and a shell covering the core material, then physically breaking or cutting at least the shell, and collecting.

In one aspect, a composition containing a cell or a cell product is produced by collecting a cell, a cell cluster or a cell product covered with a hydrogel, and comprises a solution that contains the cell, the cell cluster, and/or the cell product. The solution contains a hydrogel-derived component. A concentration of the hydrogel-derived component to the solution is 0.5 w/w % or less.

### BRIEF DESCRIPTION OF DRAWINGS

FIG. 1 is a schematic view of a string-shaped capsule according to an embodiment.

FIG. 2 is a schematic cross-sectional view of a string-shaped capsule according to an embodiment.

FIG. 3 is a schematic view for explaining a method for producing a string-shaped capsule according to an embodiment.

FIG. 4 is a graph showing the number of K562 cells harvested in Examples 1 to 3 and Reference Example 1.

FIG. 5 is a graph showing a viability of K562 cells in Examples 1 to 3 and Reference Example 1.

FIG. 6 is a microscopic image showing a state of NHDF cells cultured in a capsule in Example 4.

FIG. 7 is a microscopic image showing a state of NHDF cells when cutting a capsule in Example 4.

FIG. 8 is a microscopic image showing a state of NHDF cells after shaking of cut capsules in Example 4.

FIG. 9 is a microscopic image showing a state of NHDF cells cultured in a capsule in Example 5.

FIG. 10 is a microscopic image showing a state of NHDF cells after shaking of cut capsules in Example 5.

FIG. 11 is a graph showing the amounts of viral vectors collected in Examples 6 and 7 and Reference Examples 2 and 3.

FIG. 12 is a graph showing results of evaluating the loss of cell collection in the presence or absence of alginic acid.

### DESCRIPTION OF EMBODIMENTS

It should be noted that the present disclosure is not limited to the particular apparatus and method described. The term used in the present disclosure is for the purpose of describing particular embodiments or examples and is not intended to limit the scope of the claims. When the term “about” is used in the present specification, it means plus or minus 5% of the numerical value used.

### [Capsule]

A capsule may have a core material containing a cell or a cell cluster and a shell covering the core material. A shape of the capsule is not particularly limited. The capsule may be, for example, substantially spherical or substantially string-shaped.

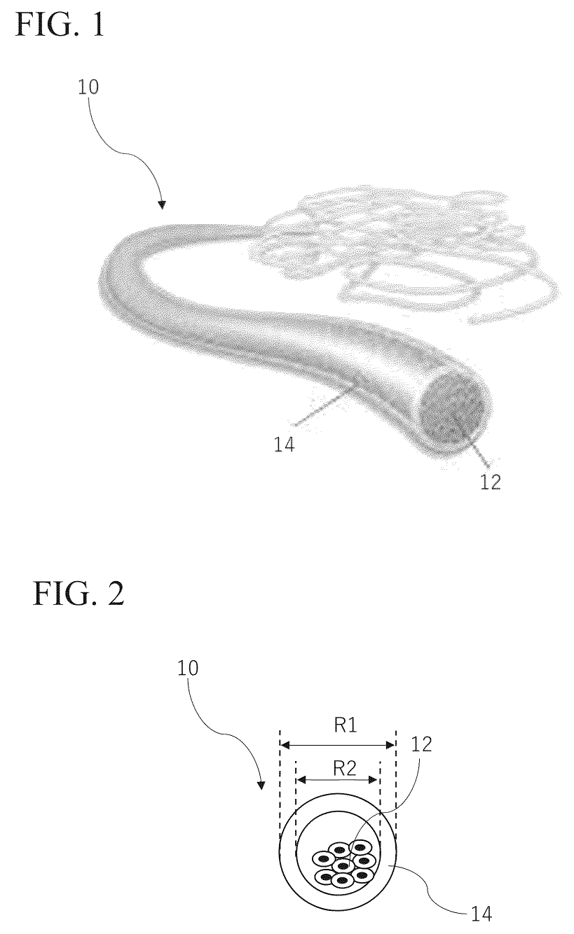

Preferably, the shape of the capsule 10 may be a substantially string shape extending continuously (see also FIGS. 1 and 2). In this case, the core material 12 has a string shape extending continuously, and the shell 14 has a continuously extending tubular shape covering the core material. Also, a cross-sectional shape of the capsule 10 is not particularly limited, and may be substantially circular or substantially elliptical. In a case where the shape of the capsule 10 is a substantially string shape extending continuously, since a large number of cells or cell clusters can be handled in a state of being accommodated in the capsule, it may be easy to handle culture and collect of cells or cell clusters and production and collect of cell products.

The core material may contain one or more cells or cell clusters. In a case where the shape of the capsule is a string shape extending continuously, the core material may contain a large number of cells or cell clusters. The cell cluster may be, for example, a cell aggregate (spheroid). The spheroid may be formed by aggregation of cells dispersed in a capsule. In the following, it should be noted that the term “cell” may refer not only to a single cell itself but also to a cell cluster in which a plurality of cells are clustered or a cell aggregate (spheroid).

Each of the core material and the shell may have only one layer or a structure of a plurality of layers. For example, the core material may be composed of a plurality of layers of materials covered with a shell. In this case, the respective layers of the core materials may be made of the same material, or may be made of different materials. Similarly, the shell covering the core materials may be composed of a plurality of layers of materials. In this case, the respective layers of the shells may be made of the same material, or may be made of different materials.

The length (maximum length in one direction) of each capsule may be, for example, 1 cm or more, preferably 5 cm or more, more preferably 10 cm or more, and still more preferably 20 cm or more. In a case where the shape of the capsule is a substantially string shape extending continuously, the lengths of the core material and the shell may be, for example, 5 cm or more, more preferably 10 cm or more, and still more preferably 20 cm or more. The longer the length of the capsule, the more cells or cell clusters can be accommodated in one capsule. From the viewpoint of ease of handling, the length of the capsule may be, for example, 200 cm or less.

An outer diameter of the shell is not particularly limited, and may be, for example, in the range of 0.2 μm to 5000 μm. Regarding the outer diameter of the shell, the outer diameter of the shell is preferably 1 μm to 2000 μm, and more preferably 10 μm to 1000 μm. Here, the outer diameter may be defined by an average value of outer diameters measured at a plurality of positions, for example, 10 positions.

If the capsule is substantially spherical, the outer diameter of the shell may be defined by a diameter of the shell. If the capsule is string-shaped, the outer diameter of the shell may be defined by a diameter of the shell in a radial direction (reference sign R1 in FIG. 2).

An inner diameter of the shell is not particularly limited, and may be, for example, in the range of 0.1 μm to 4000 μm. The inner diameter of the shell is preferably in the range of 0.8 μm to 1000 μm, more preferably 5 μm to 500 μm. Here, the inner diameter may be defined by an average value of inner diameters measured at a plurality of positions, for example, 10 positions. If the capsule is string-shaped, the inner diameter of the shell may be defined by an inner diameter of the shell in a radial direction (reference sign R2 in FIG. 2).

A thickness of the shell, that is, a difference between the outer diameter and the inner diameter of the shell, may be preferably 10 μm to 800 μm, more preferably 100 μm to 600 μm, and even more preferably 200 μm to 500 μm. Here, the difference between the outer diameter and the inner diameter may be defined by a difference between the average value of the outer diameters described listed above and the average value of the inner diameters listed above.

In addition, the capsule may be constructed by forming the core-shell structure described above. For example, the capsule may be formed into a desired shape by coupling together a plurality of substantially spherical core-shell structures. In addition, the capsule may be obtained by forming a substantially string-shaped core-shell structure into a predetermined shape. For example, the capsule may be formed into a desired shape by bundling or folding a substantially string-shaped core-shell structure. Furthermore, the capsule according to the present embodiment may be a capsule in which the core-shell structure described above is coated with another hydrogel such as an alginate gel.

The core material may be, for example, selected from the group consisting of a medium, a culture medium, a culture supernatant, a buffer solution, physiological saline, an extracellular matrix, an extracellular matrix derived from organisms or cells, a collagen solution, a laminin, agarose, nanocellulose, methylcellulose, sucrose, a polyvinyl alcohol, a serum, plasma, materials processed from serum or plasma, hyaluronic acid, a proteoglycan, elastin, pullulan, dextran, pectin, gellan gum, xanthan gum, guar gum, carrageenan, glucomannan, an alginate solution, a hydrogel, a chitosan gel, Matrigel, a collagen gel, a gelatin, a peptide gel, a fibrin gel, an alginate gel, or mixtures thereof.

The core material may contain various growth factors suitable for cell culture, cell maintenance and proliferation, cell functional expression, production of various substances by cells, or the like. The growth factors may be, for example, epidermal growth factors (EGF), platelet-derived growth factors (PDGF), transforming growth factors (TGF), insulin-like growth factors (IGF), fibroblast growth factors (FGF), nerve growth factors (NGF), vascular endothelial growth factors (VEGF), and/or hepatocyte growth factors (HGF).

When the cell contained in the core material is an adherent cell, the core material may contain a solid scaffold material if necessary. The solid scaffold material may be, for example, made of a material such as a microcarrier.

The shell may be composed of a polymer. The shell may be composed of, for example, a polymer that has sufficient permeability to cell culture medium components. The polymer may contain, for example, at least one selected from the group consisting of a hydrogel, a polysaccharide, a synthetic polymer, a polyamino acid, ornithine, lysine, arginine, histidine, guanidine, polyallylamine, polyvinylamine, polyethyleneimine, an allylamine-diallylamine copolymer, and an allylamine-maleic acid copolymer.

At least one of the core material and the shell preferably contains a hydrogel. The hydrogel is not particularly limited as long as it is a gel produced by gelating a hydrogel preparation solution (hydrogel precursor). Such a hydrogel is, for example, selected from the group consisting of an alginate gel, a chitosan gel, a collagen gel, gelatin, a peptide gel, a laminin gel, carrageenan, pectin, a gellan gum, and a fibrin gel, and mixtures thereof. The hydrogel precursor may form these hydrogels by gelation.

A chitosan gel, a collagen gel, gelatin, a peptide gel, and a laminin gel are gelled, for example, by changing temperature, pH, and/or salt concentration. Fibrinogen as a monomer acts on thrombin as an enzyme to gelate a fibrin gel. An alginate gel, carrageenan, pectin, and a gellan gum are gelled by addition of cations.

In a case where at least one of the core material and the shell contains a hydrogel, the hydrogel preferably contains an alginate gel. An alginate gel can be formed by partially cross-linking an alginic acid solution (precursor) with divalent metal ions. The alginic acid solution may be, for example, sodium alginate, potassium alginate, ammonium alginate, or a combination thereof. Also, the alginic acid may be a natural extract or may be chemically modified. Examples of the chemically modified alginic acid include methacrylate-modified alginic acid. An alginate gel has weak toxicity to cells and is suitable as a container enclosing cells.

In addition, the hydrogel may be a mixed system of the alginate described above and agar, agarose, polyethylene glycol (PEG), polylactic acid (PLA), nanocellulose, or the like. A weight of alginate with respect to a weight of a solvent of the alginic acid solution is not particularly limited, and is, for example, 0.2 wt %, preferably 0.25 wt %, and more preferably 0.5 wt %.

Examples of the divalent metal ion to be used to obtain the alginate gel include a calcium ion, a magnesium ion, a barium ion, a strontium ion, a zinc ion, and an iron ion. Preferably, the divalent metal ion is a calcium ion or a barium ion. The divalent metal ion is preferably provided to alginic acid in the form of a solution. Examples of a solution containing divalent metal ions include a solution containing calcium ions. Examples of such a solution include aqueous solutions such as an aqueous calcium chloride solution, an aqueous calcium carbonate solution, and an aqueous calcium gluconate solution. Such a solution is preferably an aqueous calcium chloride solution or an aqueous barium chloride solution. A concentration of the divalent metal ions in the solution containing divalent metal ions is, for example, 1 mM to 1M, preferably 20 to 500 mM, and more preferably 100 mM.

A raw material of the alginate gel as the hydrogel described above is preferably sodium alginate. In this case, an M/G ratio of the sodium alginate may preferably be 0.1 to 1.8. The M/G ratio is defined by a composition ratio of D-mannuronic acid and L-guluronic acid of alginates. The M/G ratio varies depending on the kind and composition of alginic acid.

In addition, a molecular weight (GPC) of sodium alginate is preferably 700,000 to 1 million, and more preferably 800,000 to 1 million.

Preferably, the shell includes a hydrogel and the core material includes a liquid or sol. Such a core-shell type capsule can be produced by a method described in any of Patent Literature 1 to 7 described above.

The cell or the cell cluster contained in the core material may include a non-adherent cell (floating cell) or may include an adherent cell. In a case where the cell or the cell cluster is an adherent cell, the shell may include a material having adhesiveness to the adherent cell. Alternatively, a material having adhesiveness to the adherent cell may be attached to an inner surface of the shell. For example, the shell may be a hydrogel such as an alginate gel, and fibrin may adhere to the inner surface of the hydrogel.

A kind of the cell is not particularly limited. Such a cell may be, for example, an ES cell or an iPS cell having differentiation pluripotency, various stem cells having differentiation multipotency (such as hematopoietic stem cells, neural stem cells, and mesenchymal stem cells), and a stem cell having differentiation unipotency (such as a liver stem cell and a germ stem cell). In addition, the cell may be various differentiated cells, for example, muscle cells such as skeletal muscle cells and cardiomyocytes, nerve cells such as cerebral cortex cells, fibroblasts, epithelial cells, hepatocytes, pancreatic β cells, and skin cells.

The cell may be a virus-producing cell, a viral vector-producing cell, an antibody-producing cell, a biopharmaceutical substance-producing cell, a biologically active substance-producing cell, an immune cell, and/or a cell for food use. The cell for food use may include at least one cell, for example, selected from adipocytes, blood cells, fibroblasts, neurons, skin cells, vascular cells, and pluripotent stem cells.

The viral vector-producing cell may be, for example, an AAV viral vector-producing cell, an adenoviral vector-producing cell, a retroviral vector-producing cell, a lentiviral vector-producing cell, a Sendai virus vector-producing cell, or a herpes simplex viral vector-producing cell. The virus-producing cell may be, for example, an oncolytic virus cell as represented by a herpes simplex virus cell, an adenovirus cell, or a vaccinia virus cell.

The “viral vector-producing cell” or “vector-producing cell” may be a cell capable of producing a viral vector or a viral vector particle. The retroviral vector-producing cell may be a “producer cell” or a “packaging cell.” One or more DNA constructs of the viral vector system may be either stably integrated into the viral vector-producing cell or maintained episomally. Alternatively, all of DNA components of the viral vector system may be transiently transfected into the viral vector-producing cells. In yet another alternative, producing cells stably expressing a portion of the components may be transiently transfected with remaining components necessary for vector production.

In a case where the cell or the cell cluster is a viral vector-producing cell, a cell product may be a viral vector. In addition, in a case where the cell or the cell cluster is a virus-producing cell, a cell product may be a virus.

The antibody-producing cell is not particularly limited, and may be, for example, a genetically modified animal cell that produces an antibody. The host cell may be, for example, a cell selected from the group consisting of CHO cells, CHO cell sublines, COS cells, Sp2/0 cells, NS0 cells, SP2 cells, PERC6 cells, YB2/0 cells, YE2/0 cells, 1R983F cells, Namalwa cells, Wil-2 cells, Jurkat cells, Vero cells, Molt-4 cells, HEK293 cells, BHK cells, HT-1080 cells, KGH6 cells, P3X63Ag8.653 cells, C127 cells, JC cells, LA7 cells, ZR-45-30 cells, hTERT cells, NM2C5 cells, and UACC-812 cells.

In a case where the cell or the cell cluster is a biopharmaceutical substance-producing cell, a cell product may be a biopharmaceutical substance. The biopharmaceutical substance may be, for example, a substance that can be used as a biopharmaceutical. The biopharmaceutical substance may be a virus, a viral vector, an antibody, or the like.

The biologically active substance-producing cell is not particularly limited, and may be, for example, a cell selected from the group consisting of an insulin secreting cell, a pancreatic islet, a pancreatic islet cell, a dopamine secreting cell, a pituitary gland cell, a growth hormone secreting cell, a parathyroid cell, a nerve growth factor secreting cell, a blood coagulation factor secreting cell, a hepatocyte, a parathyroid gland cell, and a biologically active substance expression vector (genetically modified cell).

In addition, the cell or the cell cluster may include a tissue or an organ composed of a population of cells, bacteria, fungi such as oomycetes, myxomycetes, and true fungi, and a microorganism such as algae. Examples of true fungi include koji molds and yeast.

In a case where the cell or the cell cluster is an antibody-producing cell, a cell product may be an antibody.

In a case where the cell or the cell cluster is a biologically active substance-producing cell, a cell product may be a biologically active substance. The biologically active substance may be, for example, a substance selected from the group consisting of insulin, dopamine, growth hormone, nerve growth factor, blood coagulation factor, and a biologically active substance expression vector.

A diameter of the cell or the cell cluster may be smaller than the inner diameter of the shell. Also, the diameter of the cell or the cell cluster and/or the cell product is not particularly limited, and may be, for example, 10 nm or more.

### [Method for Producing Capsule]

An example of a method for producing a capsule which includes a core material containing a cell or a cell cluster and a shell covering the core material will be described. The method for producing a capsule and including a cell or a cell cluster in the capsule is not particularly limited, and may be the same method as the method described in any of Patent Literature 1 to 7 described above.

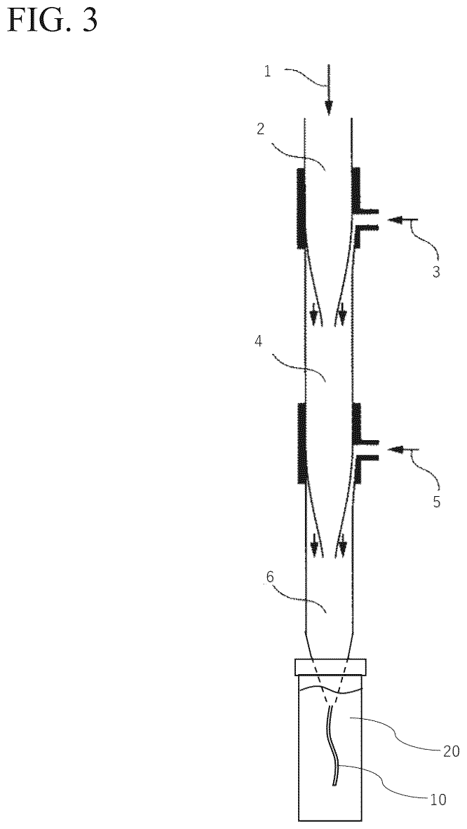

In a case where the capsule is string-shaped extending continuously, the capsule can be produced, for example, as follows (see also FIG. 3). First, while a core material (suspension) 1 containing cells is flowed, a shell precursor 3 is flowed around the suspension 1. An ingredient of the core material is as described above. In a case where the shell is a hydrogel, the shell precursor 3 may be a hydrogel precursor. The cells, the core material, and the hydrogel precursor are as described above.

Preferably, the suspension 1 is flowed as a laminar flow. This laminar flow is formed in a first introduction tube 2. The shell precursor 3 is flowed so as to cover the outer periphery of the flow of the suspension 1. That is, the shell precursor 3 flows coaxially with the suspension 1 and in the same direction. Preferably, the shell precursor 3 is flowed as a laminar flow. Thus, a flow of the shell precursor 3 surrounding the flow of the suspension 1 is formed at a second introduction tube 4.

The shell precursor 3 is then gelled or solidified to form a tubular shell covering the suspension 1. The gelation or solidification of the shell precursor 3 can be realized by bringing a sheath solution 5 containing a material that gels or solidifies the shell precursor 3 into contact with the outer periphery of the flow of the shell precursor 3. In an aspect shown in FIG. 3, the sheath solution 5 surrounds the periphery of the shell precursor (second laminar flow) 3 at a third introduction tube 6. That is, the sheath solution 5 flows coaxially with the suspension 1 and the shell precursor 3 and in the same direction.

The suspension 1, the shell precursor 3, and the sheath solution 5 may be started to flow in any order at the start, and may be stopped in any order at the stop. However, from the viewpoint of confining the cells without leakage, it is preferable to start flowing the suspension 1 last at the start of production, and it is desirable to stop the suspension 1 first at the time of stopping the production.

The suspension 1, the shell precursor 3, and the sheath solution 5 flow out of the third introduction tube 6 and are immersed, for example, in a liquid or a suspension 20 such as a physiological saline or liquid medium. Here, the shell precursor 3 flows out of the third introduction tube 6 while being gelled by application of a gelling agent. As a result, the capsule 10 containing a tubular hydrogel covering the suspension is formed.

In an aspect shown in FIG. 3, a flow of a cell suspension 1, a flow of the shell precursor 3 and a flow of the sheath solution 5 were formed, and then they are flowed out of the third introduction tube 6 to form a capsule. Alternatively, a flow of the suspension 1 and a flow of shell precursor 3 covering the outer periphery of the first laminar flow are formed, and then these flows may be discharged e into a container accommodating the sheath solution 5 containing a material that gels or solidifies the shell precursor 3. Thereby, the capsule having a similar structure can be formed. In this case, a large number of spherical capsules can also be produced by intermittently discharging the flow of the suspension 1 and the flow of shell precursor 3.

In a case where the core material contains an adherent cell, the core material may contain a solid scaffold material or a precursor of an adhesive material if necessary. The precursor of the adhesive material may include, for example, fibrinogen, components derived from bovine platelet lysate, and/or components derived from human platelet lysate (hPL). Such a component may be, for example, an insoluble component in a bovine platelet lysate or a human platelet lysate, or may be a fibrin-containing substance. Here, the fibrin-containing material may contain an insoluble component derived from plasma, platelet, or platelet lysate. In this case, the adhesive material may be precipitated from the precursor of the adhesive material in the core material. Therefore, an inner surface of the shell can be coated with an adhesive material. For example, in a case where the precursor of the adhesive material is fibrinogen, a component derived from bovine platelet lysate, and/or a component derived from human platelet lysate (hPL), fibrin can be attached to the inner surface of the shell.

### [Method for Producing Cell or Cell Cluster]

First, cells are cultured using a capsule including a core material containing a cell or a cell cluster and a shell covering the core material (culturing step). The cells are cultured in a state in which the cells are covered with a capsule. Thus, the cells, together with the capsule, may be immersed in a solution such as a liquid medium, in a state of being encased in the capsule. The conditions of cell culture are appropriately determined depending on kinds of cells to be cultured or the purpose of culture.

Next, at least the shell of the capsule is physically broken or cut. The physical breaking or cutting of the shell may be partially breaking the shell or cutting the shell into multiple parts. The breaking or cutting of the shell can be performed by piercing the shell with a needle or by cutting the shell with a cutter or scissors.

In a case where the shape of the capsule is a string shape extending continuously, it is preferable that the physical breaking or cutting of the shell be performed by cutting the string-shaped capsule into a plurality of pieces.

Next, the capsule including the physically broken or cut shell is left to stand or moved, for example, in a solution such as a medium. In this case, the cells or the cell clusters and/or the cell products are released out of the inside of the capsule. The solution may typically be a liquid medium.

In this step, preferably, the capsule including the physically broken or cut shell is moved, for example, in a solution such as a liquid medium. This operation may be repeating of aspirating/discharging of the medium that contains the capsule including the physically broken or cut shell, or may be rotating or swinging a container accommodating the medium that contains the capsule, or the like. Accordingly, the cells or the cell clusters are released out of the inside of the capsule more effectively.

In a case where the shell is physically cut, it is preferable that the shell be cut, for example, 10 times or more, 30 times or more, or 50 times or more by cutting means such as scissors or a cutter. Also, it is preferable that the majority of the cut shell (small pieces), for example, 80% or more of the plurality of small pieces, be divided into sizes having a diameter of, for example, 5 cm or less, 3 cm or less, or 1 cm or less. Here, the diameter of the small piece may be defined by the maximum length of the small piece.

Since the breaking or cutting of the shell is physically performed, it can be performed without a chemical treatment. Therefore, the cells or the cell clusters in the capsule can be collected without using a chelating agent such as ethylene diamine tetraacetic acid (EDTA) which is usually used for dissolving a hydrogel such as an alginate gel. Accordingly, the shell such as an alginate gel can be easily removed from the solution. In addition, damage to the cells or the cell clusters by the chelating agent does not is caused.

In a case where the shell includes an adhesive material having adhesiveness to the adherent cells, or in a case where the adhesive material having adhesiveness to the adherent cells adheres to the inner surface of the shell, a process of dissolving the adhesive material may be performed when collecting the cells or the cell clusters. Dissolution of the adhesive material can be achieved by adding a reagent to the solution to dissolve the adhesive material. For example, in a case where fibrin is used as the adhesive material, it is sufficient to add a reagent that selectively dissolves fibrin to a solution containing a capsule. The reagent that selectively dissolves fibrin may be, for example, a proteolytic enzyme such as nattokinase, urokinase, plasmin, fibrin/fibrinogen lytic enzyme, and trypsin. Accordingly, it is possible to make it easier to release adherent cells from physically broken or cut capsule.

The treatment of dissolving the adhesive material may be performed before or after physically breaking or cutting the shell. Even in this case, it is preferable that the treatment of dissolving the adhesive material be performed by a reagent that hardly dissolves the shell.

Next, after physically breaking or cutting at least the shell, the cell or the cell cluster are separated from the capsule including the physically broken or cut shell. This separation can be performed, for example, by filtering a solution containing capsules by using a mesh. Alternatively or additionally, a centrifugation may be used. Accordingly, it is possible to remove the capsule from a solution such as a liquid medium.

By the above process, a cell-containing composition (cell population) can be produced by collecting cells or cell clusters from the capsule.

Here, in a case where cells or cell clusters in the shell form spheroids, it is preferable that diameters of the cells or cell clusters be smaller than inner diameter of the shell. It is more preferable that the diameters of the cells or cell clusters be 90% or less of the inner diameter of the shell. Accordingly, the cells or the cell clusters can be easily released out of the capsule including the physically broken or cut shell.

The length (maximum length in one direction) of each capsule may be, for example, 1 cm or more, preferably 5 cm or more, more preferably 10 cm or more, and still more preferably 20 cm or more. In a case where the shape of the capsule is a substantially string shape extending continuously, the length of the core material and the shell may be, for example, 5 cm or more, more preferably 10 cm or more, and still more preferably 20 cm or more. In a case where the capsule has such a macroscopic size, it is easy to physically break or cut the shell. In addition, from the viewpoint of releasing the cells from the physically broken or cut shell, it is preferable that the core material contain a liquid or a sol. In other words, it is preferable that the core material be not a solid or a gel.

In a case where the shape of the capsule is a substantially string shape extending continuously, it is preferable that the capsule be cut, for example, 10 times or more, 30 times or more, or 50 times or more by cutting means such as scissors or a cutter. Also, it is preferable that the majority of the cut capsule (small pieces), for example, 80% or more of the plurality of small pieces, be divided into lengths of, for example, 5 cm or less, 3 cm or less, or 1 cm or less. Therefore, the cells or the cell clusters and/or the cell products can be released out of the inside of the capsule more efficiently.

### [Method for Producing Cell Product]

First, cells are cultured by using a capsule including a core material containing a cell or a cell cluster and a shell covering the core material (culturing step). Details of a culturing step are as described above. However, in this culturing step, the cells or the cell clusters produce cell products. That is, the cell produces a cell product in a state of being accommodated in the capsule. Here, at least a portion of the cell product is retained in the capsule. The cell product may be, for example, a virus, a viral vector, an antibody, a biopharmaceutical substance, or a biologically active substance.

Next, at least the shell of the capsule is physically broken or cut (shell breaking step). Details of the shell breaking step are as described above.

Next, the capsule including the physically broken or cut shell is left to stand or moved, for example, in a solution such as a medium. In this case, the cell product retained in the capsule is released out of the inside of the capsule. The solution may typically be a liquid medium.

In this step, preferably, the capsule including the physically broken or cut shell is moved, for example, in a solution such as a liquid medium. This operation may be shaking of the capsule including the physically broken or cut shell in a medium, and may be repeating of aspirating/discharging of the medium that contains the capsule using a pipette or needle, or the like. Accordingly, the cell product is released out of the inside of the capsule more effectively.

Since the breaking or cutting of the shell is physically performed, it can be performed without a chemical treatment. Therefore, the cell product in the capsule can be collected without dissolving a hydrogel such as an alginate gel using a chelating agent such as ethylene diamine tetraacetic acid (EDTA) or citric acid. Accordingly, the cells or the cell clusters can be easily collected from the capsule while suppressing the remaining of the hydrogel-derived substance.

Next, after physically breaking or cutting at least the shell, the cell product are separated from the capsule including the physically broken or cut shell. This separation can be performed, for example, by filtering a solution containing capsules. Accordingly, it is possible to remove the capsule from a solution such as a liquid medium.

A cell may be removed from the cell product by removing capsules including physically broken or cut shells from the cell product by filtration as described above, followed by extraction of a culture supernatant. Thereafter, the cell product-containing composition is produced by centrifuging and purifying the culture supernatant.

In a case where the cell product remains in the cell, the cell product may be extracted into a culture medium by destroying the cell before removing the cell. In this case, it is possible to suppress the loss of a portion cell products together with the removal of the cells.

In addition, in the aspect described above, the capsule including the physically broken or cut shell is removed by filtration, and then a treatment of removing the cell is performed. Alternatively, cells may also be removed by filtration along with the capsule including physically broken or cut shells.

According to the above process, a cell product can be produced. The inventors of the present application have newly found that a portion of the cell product remains in the shell, for example, a shell including a hydrogel such as an alginate gel. In particular, the inventors have newly found that a portion of an antibody, virus, or viral vector remains in a shell including a hydrogel such as an alginate gel. In the above embodiment, as described above, since the shell is physically broken or cut, the cell product remaining in the capsule can also be collected. Therefore, a collection rate of the cell product is improved.

Preferably, the shell includes a hydrogel and the core material includes a liquid or sol. In a case where the core material is a liquid or sol, by physically breaking or cutting the hydrogel as a shell, cells or cell clusters and/or cell products are easily flow out of the capsule including the broken or cut shell. Therefore, the collection rate of the cell product can be further improved.

A diameter of the cell product is not particularly limited, and may be, for example, 10 nm or more, preferably 20 nm or more, and more preferably 30 nm or more. A portion of the cell products having such sizes may remain, for example, in a shell including a hydrogel such as an alginate gel. Therefore, the method according to the embodiment can be more suitably applied.

### [Composition Containing Cell or Cell Product]

The composition containing a cell or a cell product may be produced by the above-amended method for producing a cell or the above-amended method for producing a cell product. Here, in the production method, the shell forming the capsule is removed by being broken or cut and filtered. Thus, components derived from the shell can be mostly removed from the produced cell population or cell product. From such a viewpoint, the following composition containing a cell or a cell product can be obtained.

That is, the composition containing a cell or a cell product produced by collecting a cell or a cell cluster or a cell product coated with a hydrogel may include a solution containing the cell or the cell cluster and/or the cell product. The solution contains a hydrogel-derived component. The hydrogel-derived component may be obtained by dissolving a part of the hydrogel constituting the core and/or the shell.

A concentration of the hydrogel-derived component in the solution containing the cell or the cell cluster and/or a cell product may be 0.5 w/w % or less. The concentration of the hydrogel-derived component in the solution is preferably 0.25 w/w % or less, and more preferably 0.1 w/w % or less.

The concentration of the hydrogel-derived component in the solution may be, for example, 0.000015 w/w % or more, 0.000010 w/w % or more, or 0.000005 w/w % or more.

Here, the hydrogel may be an alginate gel as described above. In this case, the hydrogel-derived component is alginic acid (not gelled). Alginic acid may slightly remain in the solution containing the cell population or cell product.

A method for measuring the concentration of the hydrogel-derived component is not particularly limited, and the concentration may be measured by a known method. As an example, the concentration of alginic acid can be measured by a carbazole method. In the carbazole method, the concentration of alginic acid in a solution is measured by quantifying uronic acid contained in alginic acid.

### Example 1

Example 1 will be described. The following cells and materials were prepared.
Cell: Human chronic myeloid leukemia-derived cells (K562 cells)
Core material: 0.3 w/v % methylcellulose solution (R&D scientific)
Alginic acid precursor: Sodium alginate (“High-G ALG300” manufactured by Kimica Corporation)
Medium: IMDM+10% FBS+0.1% Gentamicin

First, a cell suspension, an alginic acid precursor (hydrogel precursor), and a sheath solution were prepared. The sheath solution is an aqueous solution containing 100 mM calcium chloride and 3 w/v % sucrose.

The alginic acid precursor (hydrogel precursor) is a sodium alginate solution. The sodium alginate solution was produced by adding the sodium alginate described above to physiological saline and stirring. The concentration of the sodium alginate relative to the physiological saline was 1 w/v %.

The cell suspension is obtained by introducing the K562 cells into a 0.3 w/v % methylcellulose solution (solvent: the culture medium) as a core material. The K562 cells are a floating non-adherent cell. An initial density of K562 cells in the cell suspension was 2×10⁷ cells/mL. The cells introduced into the methylcellulose solution were single cell-like cells.

Using these cell suspension, alginic acid precursor (hydrogel precursor), and sheath solution, a cell population was produced based on the method for producing a capsule described above (see also FIG. 3). That is, a flow of the cell suspension, a flow of the alginic acid precursor around the flow of the cell suspension, and a flow of the sheath solution around the flow of the alginic acid precursor were formed, and these flows were discharged into physiological saline. The alginic acid precursor was crosslinked by contacting with an aqueous calcium chloride solution as a sheath solution to form a tubular alginate gel.

Accordingly, a string-shaped capsule having a core containing cells and a shell covering the core was formed in the physiological saline. The capsule covering the cells was left to stand in the physiological saline for the desired time.

Next, the cells together with the capsule were transferred to a liquid medium, and the cells were cultured in the capsule (culturing step). The cells were cultured together with the capsule in the liquid medium in a state of being enclosed in the capsule. The cells were cultured at a temperature of 37 °C for 3 days. The liquid medium used for culture is as described above.

Next, the shell of the capsule was physically cut with scissors. The bent capsule can be cut at a plurality of places at a time by a cutter. Such cutting was performed 30 times, and a large number of portions of the capsule were cut.

Next, pipetting was performed 30 times on a solution containing the capsule including the physically cut shell. Pipetting refers to the aspirating/discharging of the medium containing the capsule with a pipette. It could be confirmed by pipetting that the cells were released out of the cut shell sufficiently.

The capsules were then removed from the medium by filtering the medium containing the capsule. Accordingly, a cell population containing a large number of K562 cells was produced.

After 4 days of culture, the cell suspension containing K562 cells collected from the capsule was recovered, and the amount of alginic acid was quantified in the solution in which the capsule and the cells were separated by centrifugation. Quantification of the alginic acid was performed by a carbazole method. As a result, a concentration of the alginic acid was 12.4 μg/mL. In this case, the concentration of the alginic acid is 2.0 w/w % or less. By washing the solution containing the alginic acid and the K562 cells, a solution (cell-containing composition) containing 0.5 w/w % or less of alginic acid and the K562 cells could be easily obtained.

### Example 2

Instead of performing pipetting on a solution containing the capsule including the physically cut shell, a container accommodating the solution containing the capsule including the physically cut shell was rotated at 60 rpm for 15 minutes on a mix rotor. A cell population containing a large number of K562 cells was produced in the same manner as in Example 1 except for this operation.

### Example 3

Instead of performing pipetting on a solution containing the capsule including the physically cut shell, a container containing the solution which contains the capsule containing the physically cut shell was oscillated (shaken) at 125 rpm for 5 minutes. A cell population containing a large number of K562 cells was produced in the same manner as in Example 1 except for this operation.

### Reference Example 1

A capsule was produced in the same manner as in Example 1, and cells were cultured. Thereafter, the capsule was dissolved by placing the capsule in a 1 mM EDTA-PBS solution and leaving for 5 minutes without cutting the capsule. Cells were then collected by P1000 pipetting.

In Reference Example 1, the amount of the alginic acid was quantified with respect to the cell suspension containing K562 cells collected. Quantification of the alginic acid was performed by a carbazole method. As a result, a concentration of the alginic acid was 290 μg/mL. This was 20 times or more the concentration of the alginic acid in the solution recovered in Example 1.

### [Measurement of Number of Viable Cells]

In Examples 1 to 3 and Reference Example 1, the number of viable cells and a viability of the produced cell population were measured. The number of viable cells and the viability were measured by a trypan blue dye exclusion test method.

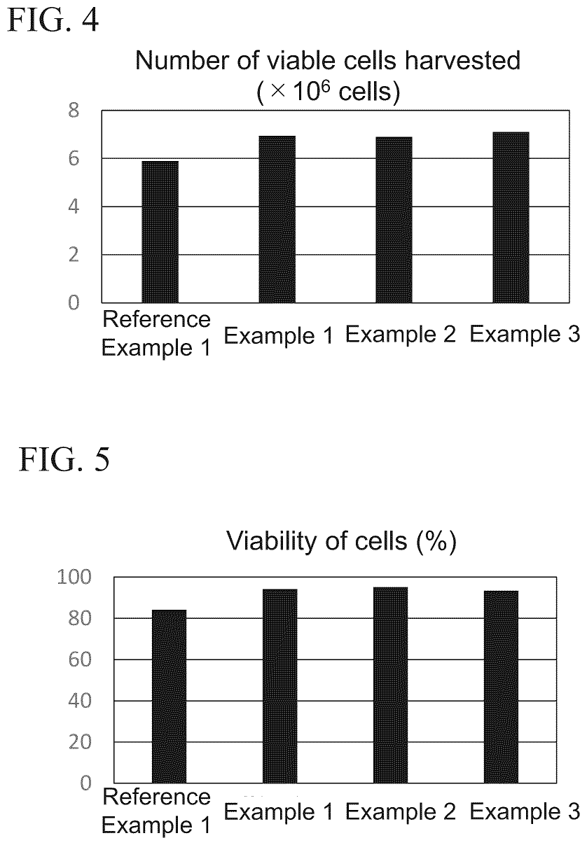

FIG. 4 is a graph showing the number of the K562 cells harvested in Examples 1 to 3 and Reference Example 1. FIG. 5 is a graph showing a viability of the K562 cells in Examples 1 to 3 and Reference Example 1.

Referring to FIG. 4, the number of harvested K562 cells produced by cutting the shell of the capsule was higher than the number of harvested K562 cells produced by dissolving the hydrogel capsule with an EDTA-PBS solution.

Furthermore, referring to FIG. 5, the viability of the K562 cells produced by cutting the shell of the capsule was higher than the viability of the K562 cells produced by dissolving the hydrogel capsule with an EDTA-PBS solution.

Regarding these results, in Examples 1 to 3, the shell was physically broken or cut and a chelating agent such as EDTA was not used. Accordingly, it is considered that the higher viability of the K562 cells was obtained because the K562 cells were not damaged by a chelating agent during collection from the capsule.

### Example 4

Example 4 will be described. The following cells and materials were prepared.
Cells: Human neonatal dermal fibroblasts (NHDF cells) Core material: 0.3 w/v % methylcellulose solution (R&D scientific)
Alginic acid precursor: Sodium alginate (“High-G ALG300” manufactured by Kimica Corporation)
Culture medium: DMEM low glucose+10% FBS+0.1% gentamicin

First, a cell suspension, an alginic acid precursor (hydrogel precursor), and a sheath solution were prepared. The sheath solution is an aqueous solution containing 100 mM calcium chloride and 3 w/v % sucrose.

The alginic acid precursor (hydrogel precursor) is a sodium alginate solution. The sodium alginate solution was produced by adding the sodium alginate described above to physiological saline and stirring. The concentration of the sodium alginate relative to the physiological saline was 1 w/v %.

The cell suspension is obtained by introducing the NHDF cells into a 0.3 w/v % methylcellulose solution (solvent: the culture medium) as a core material. The NHDF cells are adherent cells. An initial density of NHDF cells in the cell suspension was 4×10⁶ cells/mL. The cells introduced into the methylcellulose solution were single cell-like cells.

Using these cell suspension, alginic acid precursor (hydrogel precursor), and sheath solution, a cell population was produced based on the method for producing a capsule described above (see also FIG. 3). That is, a flow of the cell suspension, a flow of the alginic acid precursor around the flow of the cell suspension, and a flow of the sheath solution around the flow of the alginic acid precursor were formed, and these flows were discharged into physiological saline. The alginic acid precursor was crosslinked by contacting with an aqueous calcium chloride solution as a sheath solution to form a tubular alginate gel.

Accordingly, a string-shaped capsule having a core containing NHDF cells and a shell covering the core was formed in the physiological saline. The capsule covering the NHDF cells was left to stand in the physiological saline for the desired time.

Next, the NHDF cells together with the capsule were transferred to a liquid medium, and the NHDF cells were cultured in the capsule (culturing step). The NHDF cells were cultured together with the capsule in the liquid medium in a state of being enclosed in the capsule. The cells were cultured at a temperature of 37 °C for 2 days. The liquid medium used for culture is as described above.

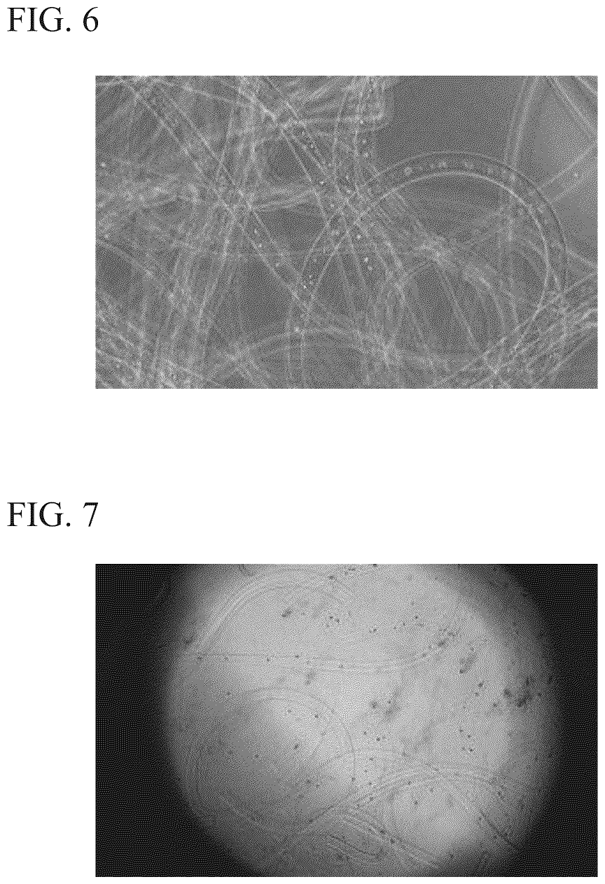

FIG. 6 is a microscopic image showing a state of K562 cells cultured in the capsule in Example 4. FIG. 6 shows a state of the capsule containing NHDF cells cultured for 2 days. As shown in FIG. 6, the NHDF cells form spheroids in a capsule. Specifically, the string-shaped capsule accommodates a large number of spheroids of NHDF cells.

The hydrogel such as an alginate gel does not substantially function as a scaffold for adherent cells such as NHDF cells. Therefore, in Example 4, it is considered that the NHDF cells formed spheroids without adhering to the shell such as an alginate gel.

Next, the shell of the capsule was physically cut with a cutter. The bent capsule can be cut at a plurality of places at a time by a cutter. Such cutting was performed 30 times, and a large number of portions of the capsule were cut (see FIG. 7).

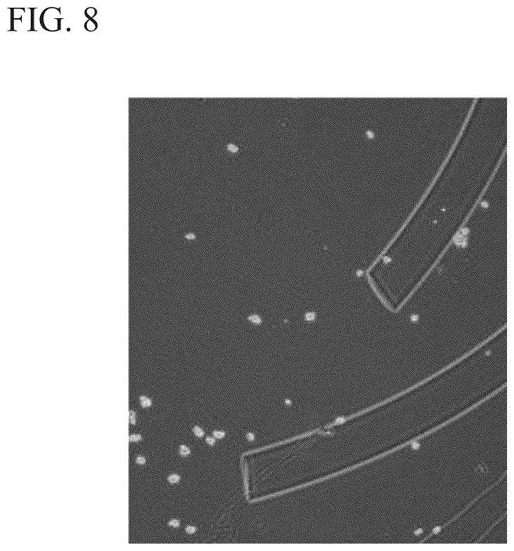

Next, a container containing the solution which contains the capsule containing the physically cut shell was oscillated (shaken) at 125 rpm for 5 minutes. Accordingly, it could be confirmed that the NHDF cells were released out of the cut shell sufficiently (see FIG. 8).

The capsules were then removed from the medium by filtering the medium containing the capsule. Accordingly, a cell population containing a large number of NHDF cell spheroids was produced.

### Example 5

Example 5 will be described. The following cells and materials were prepared.
Cells: Human neonatal dermal fibroblasts (NHDF cells)
Core material: Solution made by mixing the following medium and hPL (AventaCell, UltraGRO-PURE: HPCHXCRL50) in a 1:1 ratio.
Alginic acid precursor: Sodium alginate (“High-G ALG300” manufactured by Kimica Corporation)
Culture medium: DMEM low glucose+10% FBS+0.1% gentamicin

First, a cell suspension, an alginic acid precursor (hydrogel precursor), and a sheath solution were prepared. The sheath solution is an aqueous solution containing 100 mM calcium chloride and 3 w/v % sucrose.

The alginic acid precursor (hydrogel precursor) is a sodium alginate solution. The sodium alginate solution was produced by adding the sodium alginate described above to physiological saline and stirring. The concentration of the sodium alginate relative to the physiological saline was 1 w/v %.

The cell suspension is obtained by introducing the NHDF cells into the core material. The NHDF cells are adherent cells. An initial density of NHDF cells in the cell suspension was 2×10⁶ cells/mL. The cells introduced into the core material were single cell-like cells.

Using these cell suspension, alginic acid precursor (hydrogel precursor), and sheath solution, a cell population was produced based on the method for producing a capsule described above (see also FIG. 3). That is, a flow of the cell suspension, a flow of the alginic acid precursor around the flow of the cell suspension, and a flow of the sheath solution around the flow of the alginic acid precursor were formed, and these flows were discharged into physiological saline. The alginic acid precursor was crosslinked by contacting with an aqueous calcium chloride solution as a sheath solution to form a tubular alginate gel.

The string-shaped capsule was left to stand for a desired time while being maintained at 37 °C. Here, hPL contains fibrinogen. Fibrinogen precipitated as fibrin (adhesive material) on the inner surface of the alginate gel as a shell. Accordingly, a string-shaped capsule having a core containing NHDF cells, a shell covering the core, and fibrin adhering to an inner surface of the shell was formed.

Next, the NHDF cells together with the capsule were transferred to a liquid medium, and the NHDF cells were cultured in the capsule (culturing step). The NHDF cells were cultured together with the capsule in the liquid medium in a state of being enclosed in the capsule. The cells were cultured at a temperature of 37 °C for 2 days. The liquid medium used for culture is as described above.

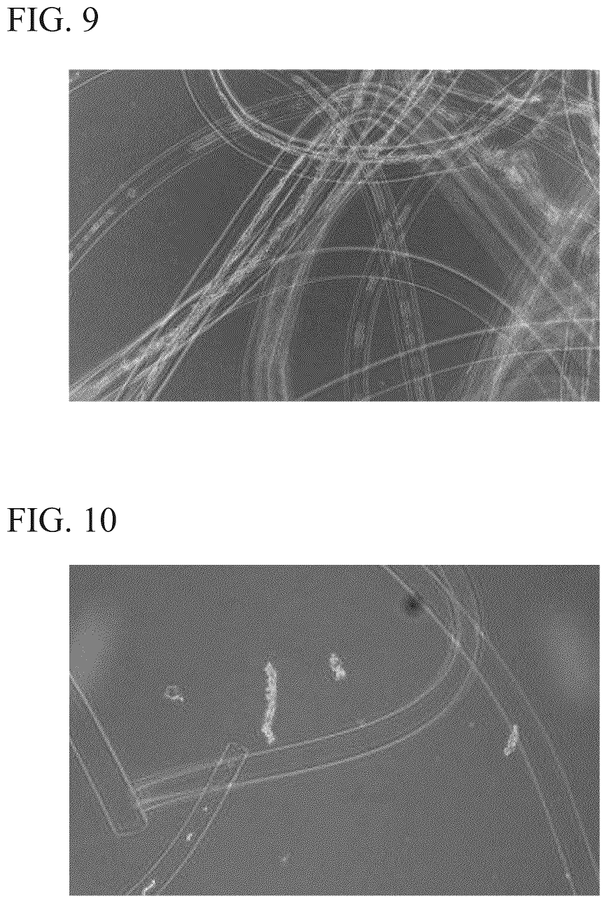

FIG. 9 is a microscopic image showing a state of NHDF cells cultured in the capsule in Example 5. FIG. 9 is a microscopic image showing a state of NHDF cells cultured in the capsule in Example 5. FIG. 9 shows a state of the capsule containing NHDF cells cultured for 2 days. As shown in FIG. 9, the NHDF cells adhere via fibrin to the shell within the capsule.

In Example 5, fibrin adheres to the inner surface of a shell containing a hydrogel such as an alginate gel. It is considered that this fibrin acts to allow NHDF cells, which are adherent cells, to adhere to an alginate gel.

Next, the shell of the capsule was physically cut with a cutter. The bent capsule can be cut at a plurality of places at a time by a cutter. Such cutting was performed 30 times, and a large number of portions of the capsule were cut.

Next, a container containing the solution which contains the capsule containing the physically cut shell was oscillated (shaken) at 125 rpm for 5 minutes. Thereafter, fibrin which is an adhesive material was dissolved. The fibrin was dissolved by adding nattokinase. Five minutes after the addition of nattokinase, a container containing the solution which contains the capsule containing the physically cut shell was further oscillated (shaken) at 125 rpm for 15 minutes. Accordingly, it could be confirmed that the NHDF cells were released out of the cut shell sufficiently (see FIG. 10).

As shown in Example 5, even if the cultured cells adhere to the adhesive material constituting the capsule, the adherent cells can be sufficiently released out of the inside of the shell by physically breaking or cutting at least the shell and by dissolving the adhesive material.

The capsules were then removed from the medium by filtering the medium containing the capsule. Accordingly, a cell population containing a large number of NHDF cell spheroids was produced.

### Example 6

Example 6 will be described. The following cells and materials were prepared.
Cell: AAV-293 (Agilent, 240073-41) Alginic acid precursor: Sodium alginate (“High-G ALG300” manufactured by Kimica Corporation)
Core material: 0.3 w/v % methylcellulose solution (R&D scientific)
Medium: DMEM+10% FBS+1% L-Glutamine (hereinafter, unless otherwise specified, the one defined on the left is referred to as a “culture medium”)
DMEM: High glucose, pyruvate (Gibco, 11995040)
Fetal Bovine Serum (CORNING, 35-010-CV, lot. 19321001)
L-Glutamine (200 mM) (Gibco, 25030081)
TrypLE select (gibco, 12563-011)
10 cm dish: cell culture dish 100 mm (Φ96×21 mm) (TPP, 93100)
1×PBS: 10×PBS (Fujifilm, 163-25265) was made 1× with MQ and used after autoclaving
pEI-pro (PPU, 115-0015)
6-well plate: multi-dish round type (6-well) (ThermoFisher, 140675)
P1000 Tips: Tips HRC UNV 1000 μL 768C/8 (RC research grade tip, general-purpose 1000 μL) (Mettler-Toledo Rainin, 30374656)
G18 needle: Terumo non-bevel needle 18G (Terumo, NN-1838N)
10 mL syringe: Terumo syringe 10 mL lock (Terumo, SS-10LZ)
AAVpro: Titration Kit (for Real Time PCR) Ver. 2 (Takara, 6233)

First, GFP, AAV1, and Helper plasmids were each added to the pEI-pro solution, and this was used as a transfection solution. The cell supernatant was then removed and a transfection solution was added to the cells (AAV-293) with a fresh medium. Cells were transfected by incubating overnight.

Next, a string-shaped capsule was prepared. First, a cell suspension, an alginic acid precursor (hydrogel precursor), and a sheath solution were prepared. The sheath solution is an aqueous solution containing 100 mM calcium chloride and 3 w/v % sucrose.

The alginic acid precursor (hydrogel precursor) is a sodium alginate solution. The sodium alginate solution was produced by adding the sodium alginate described above to physiological saline and stirring. The concentration of the sodium alginate relative to the physiological saline was 0.5 w/v %.

The cell suspension was obtained by introducing the transfected cells into a 0.6 w/v % methylcellulose solution (solvent: the culture medium) as a core material. The transfected cells are cells that produce a viral vector (AAV). An initial density of the transfected cells in the cell suspension was 3×10⁶ cells/100 μL.

Using these cell suspension, alginic acid precursor (hydrogel precursor), and sheath solution, a capsule was produced based on the method for producing a capsule described above (see also FIG. 3). That is, a flow of the cell suspension, a flow of the alginic acid precursor around the flow of the cell suspension, and a flow of the sheath solution around the flow of the alginic acid precursor were formed, and these flows were discharged into physiological saline. The alginic acid precursor was crosslinked by contacting with an aqueous calcium chloride solution as a sheath solution to form a tubular alginate gel.

Accordingly, a string-shaped capsule having a core containing cells and a shell covering the core was formed in the physiological saline. The capsule covering the cells was left to stand in the physiological saline for the desired time.

Next, the cells together with the capsule were transferred to a liquid medium, and the cells were cultured in the capsule (culturing step). The cells were cultured together with the capsule in the liquid medium in a state of being enclosed in the capsule. The cells were cultured at a temperature of 37 °C for 3 days (culturing step). The liquid medium used for culture is as described above. During this culture, viral vectors were produced from the cells.

Next, the shell of the capsule was physically cut with a cutter. The bent capsule can be cut at a plurality of places at a time by scissors. Such cutting was performed 10 times, and a large number of portions of the capsule were cut.

Next, the medium containing the cut capsule was then pipetted 10 times using the tip of P1000. 0.5 mL of the solution after pipetting was recovered in a 1.5 mL tube. The culture supernatant (cell product) was used as Example 6.

### Example 7

Example 7 will be described. In Example 7, an additional step was performed with respect to Example 6. The preparation of the cells, the production of the capsule, the cultivation of the cells, the cutting of the capsule and the pipetting of the medium containing the cut capsules were carried out exactly as in Example 6. In Example 7, using the solution after pipetting in Example 6, the solution is suspended 10 times using a G18 needle attached to the 10 ML syringe. This suspension of the solution is carried out by a process of drawing the whole amount of the solution up and exhaling the whole amount thereof with the G18 needle. The process was performed 10 times. The solution (cell product) obtained after the process was used as Example 7.

### Reference Example 2

In Example 6, the cells together with the capsule were transferred to a liquid medium, and the cells were cultured in the capsule (culturing step), and 0.5 mL of the culture supernatant was recovered in a 1.5 mL tube. The culture supernatant (cell product) was used as Reference Example 2.

### Reference Example 3

Cells transfected in Example 6 were seeded onto culture dishes without being placed in a capsule and cultured in the same liquid medium for 3 days under the same conditions. 0.5 mL of the culture supernatant after culture was collected in a 1.5 mL tube. The culture supernatant (cell product) was used as Reference Example 3.

### [Titer Evaluation]

DNase I was added to the culture supernatants acquired in Examples 6 and 7 and Reference Examples 2 and 3, and the free genome and plasmid DNA of the culture supernatant were removed.

Next, the mixture is heat-treated to deactivate DNase I. Then, Lysis Buffer was added to the mixture to elute the vector genome.

The amount (titer) of the viral vector was determined by real-time PCR using the solution from which the genome of the vector was eluted.

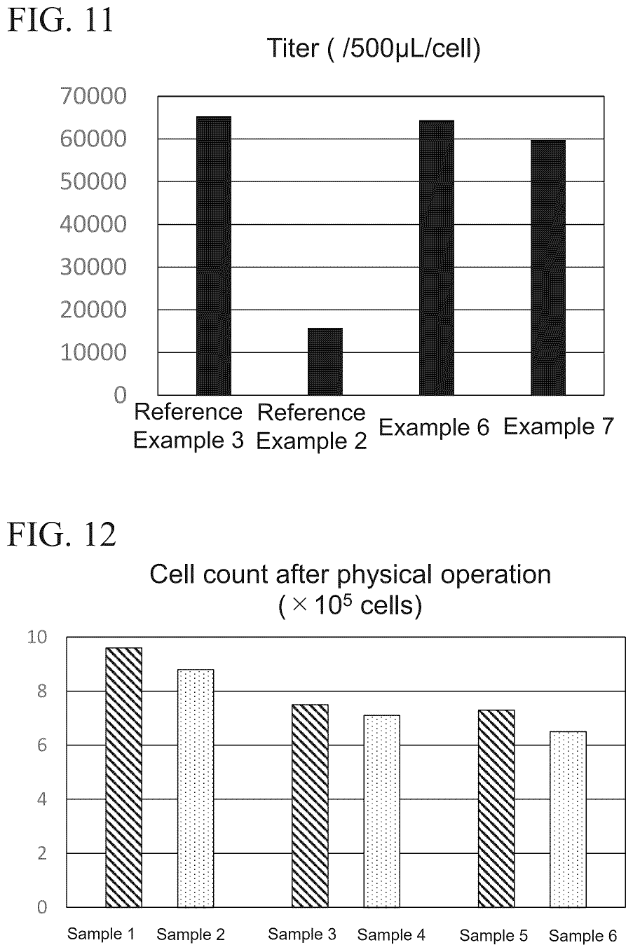

FIG. 11 is a graph showing the amounts (titers) of viral vectors in Examples 6 and 7 and Reference Examples 2 and 3. In Examples 6 and 7, the viral vector was produced in a state in which the cells were covered with the capsule, but the viral vector similar to that in Reference Example 3 cultured without being covered with the capsule could be collected.

In Reference Example 2, the culture supernatant was recovered without breaking or cutting the capsule. In Reference Example 2, the titer was smaller than those in Examples 6 and 7. This is considered to be due to a decrease in the recovery amount of the viral vector. That is, the viral vector could be collected more effectively by breaking or cutting the capsule and performing recovery.

As described above, the inventors of the present application have arrived at a process of breaking or cutting the shell constituting the capsule by finding that a portion of the cell product may be confined in the capsule. Therefore, as described above, the cell product can be collected more efficiently. In addition, since the cell product can be collected without dissolving the shell constituted by a gel such as alginate gel, alginic acid can be easily removed from a collected product (cell product-containing composition).

### [Evaluation of Loss of Cell Collection]

Next, a suspension in which alginic acid (ALG300) was added to physiological saline containing T cells at a ratio as shown in Table 1 below was produced, and the following operation was performed on the suspension, and then the number of remaining cells was examined.

The operations performed on the suspension are the following two kinds.
Operation 1: Accommodating suspension in a 15 mL tube and rolling the tube.
Operation 2: Pipetting the suspension.

TABLE 1

| | | Alginate concentration | |
|---|---|---|---|
| | Cell | (w/w %) | Operation |
| Sample 1 | T cell | 0.00 | Operation 1 |
| Sample 2 | T cell | 0.00 | Operation 2 |
| Sample 3 | T cell | 0.25 | Operation 1 |
| Sample 4 | T cell | 0.25 | Operation 2 |
| Sample 5 | T cell | 0.50 | Operation 1 |
| Sample 6 | T cell | 0.50 | Operation 2 |

The number of viable cells was counted for each sample after the above operation 1 or operation 2. Counting the number of viable cells was performed using a hemocytometer with trypan blue staining. FIG. 12 is a graph showing results of evaluating the loss of cell collection in the presence or absence of alginic acid.

Referring to FIG. 12, regardless of the methods of the physical operations 1 and 2, as the concentration of alginic acid is higher, loss of collection of T cells occurs. This is considered to be due to the fact that the viscosity of the solution containing cells increases as the concentration of alginic acid increases. That is, it may be due to the fact that cells adhere to the container together with alginic acid or are not effectively separated by centrifugation according to the increase in viscosity of the solution. Therefore, it is desirable that the concentration of alginic acid (hydrogel) in the solution be as low as possible during the collection of the cells from the solution.

In the above-described embodiments and examples, most of the hydrogel such as alginate gel constituting the capsule is not dissolved by the chelating agent and is removed by filtration. Thus, the amount of alginic acid remaining in the collected cell population and cell product can be extremely small. Therefore, it is considered that the loss during the collection of the cells or cell products as described above can be reduced.

It should be noted that at least the following inventions are specified in the present specification by the description of the foregoing embodiments and/or examples.
[Appendix 1] A method for producing a cell or a cell cluster and/or a cell product, comprising:
   culturing a cell or a cell cluster in a core material to obtain a cell, a cell cluster and/or a cell product by using a capsule including the core material and a shell covering the core material; and
   physically breaking or cutting at least the shell to collect the cell or the cell cluster and/or the cell product.
[Appendix 2] The method according to appendix 1, wherein at least one of the core material and the shell contains a hydrogel.
[Appendix 3] The method according to appendix 2, wherein the hydrogel includes an alginate gel.
[Appendix 4] The method according to any one of appendices 1 to 3, wherein the shell contains a hydrogel, and the core material contains a liquid or a sol.
[Appendix 5] The method according to any one of appendices 1 to 4, further comprising leaving or moving a capsule in a solution, the capsule including the shell physically broken or cut.
[Appendix 6] The method according to any one of appendices 1 to 5, comprising removing the capsule from a solution by filtering the solution including the capsule after physically breaking or cutting at least the shell.
[Appendix 7] The method according to any one of appendices 1 to 6, wherein the cell or the cell cluster includes a non-adherent cell.
[Appendix 8] The method according to any one of appendices 1 to 6, wherein the cell or the cell cluster includes an adherent cell.
[Appendix 9] The method according to appendix 8, wherein
   the capsule includes an adhesive material having adhesiveness to the adherent cell, and
   the method further comprises dissolving the adhesive material before or after physically breaking or cutting at least the shell.
[Appendix 10] The method according to appendix 9, wherein
   the adhesive material is fibrin, and
   the method further comprises dissolving the fibrin before or after physically breaking or cutting at least the shell.
[Appendix 11] The method according to any one of appendices 1 to 10, wherein the cell or the cell cluster includes at least one of a virus-producing cell, a viral vector-producing cell, an antibody-producing cell, a biopharmaceutical substance-producing cell, a biologically active substance-producing cell, a stem cell, a cell differentiated from a stem cell, an immune cell, and a cell for food use.
[Appendix 12] The method according to any one of appendices 1 to 10, wherein a cell or a cell cluster to be cultured includes at least one selected from the group consisting of a single cell, an isolated cell, a spheroid, a fungus, a microorganism, a tissue, and an organ.
[Appendix 13] A composition containing a cell or a cell product, the composition being produced by culturing a cell or a cell cluster in a core material to obtain a cell, a cell cluster and/or a cell product by using a capsule including the core material and a shell covering the core material, then physically breaking or cutting at least the shell, and collecting.
[Appendix 14] A composition containing a cell or a cell product, the composition being produced by collecting a cell, a cell cluster or a cell product covered with a hydrogel, and comprising:
   a solution that contains the cell, the cell cluster, and/or the cell product,
   wherein the solution contains a hydrogel-derived component, and
   a concentration of the hydrogel-derived component to the solution is 0.5 w/w % or less.
[Appendix 15] The composition containing a cell or a cell product according to Appendix 14, wherein
   the hydrogel is an alginate gel, and
   the hydrogel-derived component is alginic acid.
[Appendix 16] The composition containing a cell or a cell product according to Appendix 14 or 15, wherein the concentration of the hydrogel-derived component to the solution is 0.000005 w/w % or more.

As described above, the cell to be cultured in the supplementary notes are not limited to one cell, that is, an isolated cell or a single cell, and may include a cell cluster such as a spheroid, a tissue, or an organ. In addition, the cell or the cell cluster obtained by culture is not limited to an isolated cell or a single cell, and may include a cell cluster such as a spheroid, a tissue, or an organ. However, the cell or the cell cluster to be cultured and/or the cell or the cell cluster obtained by culturing is preferably an isolated cell, a spheroid, a fungus, and/or microorganisms. Examples of the type of the isolated cell and the cell constituting the spheroid are as described above.

As described above, the contents of the present invention have been disclosed through the embodiments, but it should not be understood that the description and the drawings constituting a part of the disclosure limit the present invention. From this disclosure, various alternative embodiments, examples, and operational techniques will become apparent to those skilled in the art. Therefore, the technical scope of the present invention is defined only by the matters specifying the invention according to the claims appropriate from the above description.

This application claims priority based on Japanese Patent Application No. 2022-190850 filed on Nov. 29, 2022, the entire contents of which are incorporated herein by reference.

1. A method for producing a cell or a cell cluster and/or a cell product, comprising:
culturing a cell or a cell cluster in a core material to obtain a cell, a cell cluster and/or a cell product by using a capsule including the core material and a shell covering the core material; and
physically breaking or cutting at least the shell to collect the cell or the cell cluster and/or the cell product.

2. The method according to claim 1, wherein at least one of the core material and the shell contains a hydrogel.

3. The method according to claim 2, wherein the hydrogel includes an alginate gel.

4. The method according to any one of claim 1,
wherein the shell contains a hydrogel, and
the core material contains a liquid or a sol.

5. The method according to any one of claim 1, further comprising leaving or moving a capsule in a solution, the capsule including the shell physically broken or cut.

6. The method according to any one of claim 1, comprising removing the capsule from a solution by filtering the solution including the capsule after physically breaking or cutting at least the shell.

7. The method according to any one of claim 1, wherein the cell or the cell cluster includes a non-adherent cell.

8. The method according to any one of claim 1, wherein the cell or the cell cluster includes an adherent cell.

9. The method according to claim 8, wherein
the capsule includes an adhesive material having adhesiveness to the adherent cell, and
the method further comprises dissolving the adhesive material before or after physically breaking or cutting at least the shell.

10. The method according to claim 9, wherein
the adhesive material is fibrin, and
the method further comprises dissolving the fibrin before or after physically breaking or cutting at least the shell.

11. The method according to any one of claim 1, wherein the cell or the cell cluster includes at least one of a virus-producing cell, a viral vector-producing cell, an antibody-producing cell, a biopharmaceutical substance-producing cell, a biologically active substance-producing cell, a stem cell, a cell differentiated from a stem cell, an immune cell, and a cell for food use.

12. The method according to any one of claim 1, wherein a cell or a cell cluster to be cultured includes at least one selected from the group consisting of a single cell, an isolated cell, a spheroid, a fungus, a microorganism, a tissue, and an organ.

13. A composition containing a cell or a cell product, the composition being produced by culturing a cell or a cell cluster in a core material to obtain a cell, a cell cluster and/or a cell product by using a capsule including the core material and a shell covering the core material, then physically breaking or cutting at least the shell, and collecting.

14. A composition containing a cell or a cell product, the composition being produced by collecting a cell, a cell cluster or a cell product covered with a hydrogel, and comprising:
a solution that contains the cell, the cell cluster, and/or the cell product,
wherein the solution contains a hydrogel-derived component, and
a concentration of the hydrogel-derived component to the solution is 0.5 w/w % or less.

15. The composition containing a cell or a cell product according to claim 14, wherein
the hydrogel is an alginate gel, and
the hydrogel-derived component is alginic acid.

16. The composition containing a cell or a cell product according to claim 14, wherein the concentration of the hydrogel-derived component to the solution is 0.000005 w/w % or more.

17. The method according to any one of claim 1, wherein
physically breaking or cutting at least the shell is performed by a needle, a cutter or scissors.

18. The method according to any one of claim 1, wherein
physically breaking or cutting at least the shell is performed without an agent for dissolving the shell.

19. The method according to any one of claim 1, wherein
physically breaking or cutting at least the shell comprises cutting the shell into a plurality of pieces.
